(19) United States
(12) Patent Application Publication
(10) Pub. No.: US 2004/0170608 A1
(43) Pub. Date: Sep. 2, 2004

(54) Use of a lentiviral vector in the treatment of pain

(76) Inventors: Rob Barber, Oxford (GB); Mimoun Azzouz, Oxford (GB); Nicholas D. Mazarakis, Oxford (GB); Susan Kingsman, Oxford (GB)

Correspondence Address: FROMMER LAWRENCE & HAUG, 745 FIFTH AVENUE- 10TH FL., NEW YORK, NY, 10151, US

(21) Appl. No.: 10/799,284

(22) Filed: Mar. 12, 2004

Related U.S. Application Data
(63) Continuation-in-part of application No. PCT/GB02/04169, filed on Sep. 12, 2002.

(30) Foreign Application Priority Data
Sep. 14, 2001 (GB) 0122237.1
May 8, 2002 (GB) 0210575.7

(51) Int. Cl. A61K 48/00; C12N 15/867

(52) U.S. Cl. 424/93.2; 435/456

(57) ABSTRACT

Provided is a method for treating and/or preventing pain, in which a vector system is administered such that an EOI is delivered to a DRG of the subject. Also provided is a method for delivering an EOI to the spinal cord using such a vector system. Further provided is a method for identifying and/or validating an EOI by delivering a test EOI to target cell; analyzing the effect of the EOI on the target cell; and selecting an EOI with therapeutic potential. An EOI identified or validated by such a method, useful in the prevention and/or treatment of pain, is thereby provided as well.

### CROSS REFERENCE TO RELATED APPLICATIONS

This application is a continuation-in-part of International application No. PCT/GB02/04169, filed on Sep. 12, 2002, published as WO 03/025188 on Mar. 27, 2003, and claiming priority to GB application Nos. 0122237.1, filed on Sep. 14, 2001 and 0210575.7, filed on May 8, 2002.

This application makes reference to U.S. application Ser. No. 10/716,725, filed on Nov. 19, 2003, which is a continuation-in-part of U.S. application Ser. No. 10/429,608, filed on May 5, 2003, which is a continuation-in-part of International application no. PCT/GB01/04866, filed on Nov. 2, 2001, published on May 10, 2002 as WO 02/36170, and claiming priority to GB application nos. 0026943.1, filed on Nov. 3, 2000, 0102339.9, filed on Jan. 30, 2001 and 0122238.9 filed on Sep. 14, 2001. U.S. application Ser. No. 10/716,725 is also a continuation-in-part of International application no. PCT/GB03/00426, filed on Oct. 3, 2003, and claiming priority to GB application nos. 0223076.1, filed on Oct. 4, 2002, 0228314.1, filed on Dec. 4, 2002 and 0318213.6, filed on Aug. 4, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a vector system. In particular, the present invention relates to a vector system capable of delivering an entity of interest (“EOI”)—such as a nucleotide sequence of interest (“NOI”)—to a cell.

The present invention also relates to the use of such a vector system in a method for treating and/or preventing pain in a subject. In the method, the vector system is administered such that the EOI is delivered directly or indirectly to one or more of the dorsal root ganglia (DRG) of the subject.

The present invention also relates to the use of such a vector system in a method for identifying and/or validating an EOI useful in pain relief. In this method, the EOI is introduced to a target cell (such as a DRG cell) either in vitro or in vivo within a subject.

### BACKGROUND OF THE INVENTION

Production of an electrical signal in a cell is dependent on two basic features of the plasma membrane of excitable cells: the existence of a resting membrane potential and the presence of specific ion channels. The resting membrane potential is an electrical voltage difference across the membrane. The ion channels in the membrane open (or close) in response to specific stimuli, allowing specific ions to diffuse across the plasma membrane down their electrochemical gradient. The result is a flow of current, which can change the membrane potential of the cell.

There are many instances in which it is desirable to modulate the membrane potential of an excitable cell. For example, pain is transmitted from the periphery into the central nervous system via a sensory nerve impulse. Modulation of the excitability of the sensory neuron responsible provides an approach to control pain.

The conventional method for treating pain is the administration of drugs such as anaesthetics, capsaisin, NSAIDs, opioids, NMDA antagonists and dorsal horn inhibitors. A recent survey showed that at least 40% of patients do not get adequate relief using such drugs.

For the treatment of chronic pain, long term treatment is necessary, entailing indefinite frequent repeat doses of the drug, which is inconvenient and expensive.

The systemic administration of drugs needed only in small areas of the body is also associated with many side effects.

It is therefore desirable to develop a new approach to the treatment of pain, in particular chronic intransient pain, which has greater efficacy, specificity and possibly reduces the frequency and/or number of repeat treatments.

### SUMMARY OF THE INVENTION

In a broad aspect, the present invention relates to method for treating and/or preventing pain in a subject. The method involves administering a vector system that is capable of delivering an entity of interest (“EOI”) to a DRG.

Cell bodies of sensory neurons are found in the DRG. The vector system may thus be capable of delivering the EOI to a sensory neuron.

In a first preferred aspect the EOI is capable of modulating the cellular excitability of a target cell, for example a sensory neuron.

In a second preferred aspect the EOI is capable of modulating the expression or activity of a receptor, such as an opioid receptor or an NMDA receptor.

In a third preferred aspect the EOI is capable of encoding a neurotrophic factor, such as glial cell-derived neurotrophic factor (GDNF)

The vector system can be a non-viral system or a viral system, or combinations thereof. In addition, the vector system itself can be administered by viral or non-viral techniques.

In a first preferred embodiment, the vector system is administered directly to the DRG of a subject, for example by direct injection.

Direct administration to the DRG has the advantage that, since the administration site is the same as the target site, there are no side effects associated with delivery of the EOI to the administration site and surrounding tissue.

In a second preferred embodiment the vector system is administered to a site which is distant to the DRG. The vector system (or part thereof) then travels to the DRG by retrograde transport.

In this embodiment, preferably the vector system is or comprises at least a part of an entity which causes the system to travel by retrograde transport. The entity may be rabies G protein (glycoprotein) or a mutant, variant, homologue or fragment thereof.

In non-viral vector systems of this aspect of present invention, the at least part of the rabies G protein (or a mutant, variant, homologue or fragment thereof) may be used to encapsulate or enshroud an EOI. Thus the at least part of the rabies G protein (or a mutant, variant, homologue or fragment thereof) may form a matrix around the EOI. Here, the matrix may contain other components—such as a liposome type entity.

In viral vector systems of this aspect of present invention, typically the vector system is pseudotyped with at least a part of a rabies G protein or a mutant, variant, homologue or fragment thereof.

Administration to a site which is distant to the DRG is advantageous because such a site may be more accessible than the DRG. Also, by using retrograde transport is it possible to deliver the EOI to certain cells or groups of cells. For example, where the vector system is administered peripherally at the site of pain, the vector system (or part thereof) will travel to the DRG by retrograde transport and deliver the EOI to cells which are directly involved in sensing the pain.

In further broad aspects, the present invention relates to:

(i) the use of such a vector system in the manufacture of a pharmaceutical composition to treat and/or prevent pain;

(ii) a method for analysing the effect of an entity of interest in a cell (such as a sensory neuron) using such a vector system;

(iii) a method for analysing the function of a gene or protein using such a vector system;

(iv) a cell (such as a DRG or sensory neuron) which has received an EOI from such a vector system.

The present invention also relates to a method for delivering an EOI to the spinal cord, which comprises the following steps:

(i) delivery of an EOI to the cell body of a sensory neuron using a vector system according to the second preferred embodiment of the present invention;

(ii) optional modification of the EOI; and

(iii) delivery of the optionally modified EOI from the cell body of the sensory neuron to the spinal cord via the central branch of the sensory neuron.

In a further broad aspect, the present invention relates to methods for the discovery of novel treatments for pain.

In particular, the present invention provides a method for identification and/or validation of an EOI useful in the prevention and/or treatment of pain. Preferably the method comprises the following steps

(i) delivery of a test EOI to target cell;

(ii) analysis of the effect of the EOI on the target cell; and

(iii) selection of an EOI with therapeutic potential.

As used herein, the term “treatment” includes curative effects, alleviation effects, and prophylactic effects. The target cell may be in vivo or in vitro. Preferably the target cell is derivable from a DRG. For example, the target cell may be a cell within a DRG in situ, or a cultured DRG-derived cell (such as a cell within a dissociated or explant culture).

Analysis of the effect of the EOI on the target cell may involve monitoring EOI-induced modulation of the transcriptome and/or proteotome of the target cell. In this way novel genes may be identified as a result of their capacity to modulate the transcriptome/proteotome or as a result of EOI-induced modulation of their transcription/translation.

The present invention also provides an in vivo method for screening an EOI. The method may involve

(i) administration of a vector system such that it delivers an EOI to a DRG of a subject by the method as described in the first broad aspect of the invention; and

(ii) analysis of pain in the subject.

Most preferred is a method which involves in vitro screening for an EOI with therapeutic potential, followed by in vivo verification of its therapeutic effect.

In a further aspect, the present invention relates to an EOI identified by the method of the present invention. Preferably the EOI (or a derivative or product thereof) is useful in pain relief. For example, the EOI may be capable of completely or partially blocking the transmission and/or perception of pain.

### BRIEF DESCRIPTION OF THE DRAWINGS

The following Detailed Description, given by way of example, but not intended to limit the invention to specific embodiments described, may be understood in conjunction with the accompanying drawings. Various preferred features and embodiments of the present invention are described by way of non-limiting example and with reference to the accompanying drawings in which:

FIGS. 1A and 1B show dissociated DRG transduced with pONY8G,5′cPPT at MOI=10 at DIV4. FIG. 1A shows expression of GFP and FIG. 1B shows the corresponding bright field image.

FIGS. 2A-2E show viral transfer of genes to sensory neurons. Expression of the reporter gene β-galactosidase in the dorsal root is depicted in FIGS. 2A-2C and expression in the DRG is depicted in FIGS. 2D and 2E, after injection of pONY8Z pseudotyped with rabies-G into the dorsal horn of the spinal cord. Sections show immunofluorescence for β-galactosidase 5 weeks after viral injections. Expression of β-gal is detectable in Schwann cells, axons (arrowheads) and DRG neurons (arrow). Magnification is indicated in each panel.

FIGS. 3A-3E show expression of the reporter gene β-galactosidase in DRG neurons 4 weeks after pONY8Z vectors pseudotyped with rabies G envelope were injected into the footpad of 4 rats.

FIGS. 4A-4C show transduction of DRG neurons after intranerval injection of pONY8Z rabies-G. Sections show immunofluorescence for β-galactosidase (FIG. 4A) and the neuronal marker NeuN (FIG. 4B) 5 weeks after viral injections. FIG. 4C shows the composite image.

### DETAILED DESCRIPTION OF THE INVENTION

The present invention relates to a new use of a vector system.

As used herein the term “vector system” includes any vector that is capable of infecting or transducing or transforming or modifying a recipient cell with an EOI.

The vector system can be a non-viral system or a viral system.

Non-Viral Vector Systems

Non-viral delivery systems include but are not limited to DNA transfection methods. Here, transfection includes a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1, 2, -bis (oleoyloxy)-3-(trimethylammonio) propane (DOTAP)-cholesterol complexes (Wolff and Trubetskoy 1998 Nature Biotechnology 16: 421) and combinations thereof.

Viral Vector Systems

The vector system may be a viral vector system. Viral vector or viral delivery systems include but are not limited to adenoviral vectors, adeno-associated viral (AAV) vectors, herpes viral vectors, retroviral vectors (including lentiviral vectors) and baculoviral vectors.

Preferably the vector system is a retroviral vector system.

Retroviruses

The concept of using viral vectors for gene therapy is well known (Verma and Somia (1997) Nature 389:239-242).

There are many retroviruses. For the present application, the term “retrovirus” includes: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al (“Retroviruses” 1997 Cold Spring Harbour Laboratory Press Eds: J M Coffin, S M Hughes, H E Varmus pp 758-763).

In a preferred embodiment, the retroviral vector system is derivable from a lentivirus. Lentiviruses also belong to the retrovirus family, but they can infect both dividing and non-dividing cells (Lewis et al (1992) EMBO J. 3053-3058).

The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). In a preferred embodiment, the retroviral vector system is derivable from EIAV.

Details on the genomic structure of some lentiviruses may be found in the art. By way of example, details on HIV and EIAV may be found from the NCBI Genbank database (i.e. Genome Accession Nos. AF033819 and AF033820 respectively).

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and other factors required to make more virus, which can leave the cell by a process sometimes called “budding”.

Each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′end of the RNA. The sizes of the three elements can vary considerably among different retroviruses.

For the viral genome, the site of transcription initiation is at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Retroviruses may also contain “additional” genes which code for proteins other than gag, pol and env. Examples of additional genes include in HIV, one or more of vif, vpr, vpx, vpu, tat, rev and nef. EIAV tat, rev and S2.

Proteins encoded by additional genes serve various functions, some of which may be duplicative of a function provided by a cellular protein. In EIAV, for example, tat acts as a transcriptional activator of the viral LTR. It binds to a stable, stem-loop RNA secondary structure referred to as TAR. Rev regulates and co-ordinates the expression of viral genes through rev-response elements (RRE). The mechanisms of action of these two proteins are thought to be broadly similar to the analogous mechanisms in the primate viruses. The function of S2 is unknown. In addition, an EIAV protein, Ttm, has been identified that is encoded by the first exon of tat spliced to the env coding sequence at the start of the transmembrane protein.

As used herein the term “vector system”, when referring to a viral vector system also includes a vector particle capable of transducing a recipient cell with an NOI.

A vector particle includes the following components: a vector genome, which may contain one or more NOIs, a nucleocapsid encapsidating the nucleic acid, and a membrane surrounding the nucleocapsid.

The term “nucleocapsid” refers to at least the group specific viral core proteins (gag) and the viral polymerase (pol) of a retrovirus genome. These proteins encapsidate the packagable sequences and are themselves further surrounded by a membrane containing an envelope glycoprotein.

Once within the cell, the RNA genome from a retroviral vector particle is reverse transcribed into DNA and integrated into the DNA of the recipient cell.

The term “vector genome” refers to both to the RNA construct present in the retroviral vector particle and the integrated DNA construct. The term also embraces a separate or isolated DNA construct capable of encoding such an RNA genome. A retroviral or lentiviral genome should comprise at least one component part derivable from a retrovirus or a lentivirus. The term “derivable” is used in its normal sense as meaning a nucleotide sequence or a part thereof which need not necessarily be obtained from a virus such as a lentivirus but instead could be derived therefrom. By way of example, the sequence may be prepared synthetically or by use of recombinant DNA techniques. Preferably the genome comprises a psi region (or an analogous component which is capable of causing encapsidation).

The viral vector genome is preferably “replication defective” by which we mean that the genome does not comprise sufficient genetic information alone to enable independent replication to produce infectious viral particles within the recipient cell. In a preferred embodiment, the genome lacks a functional env, gag or pol gene. In a highly preferred embodiment the genome lacks env, gag and pol genes.

The viral vector genome may comprise some or all of the long terminal repeats (LTRs). Preferably the genome comprises at least part of the LTRs or an analogous sequence which is capable of mediating proviral integration, and transcription. The sequence may also comprise or act as an enhancer-promoter sequence.

It is known that the separate expression of the components required to produce a retroviral vector particle on separate DNA sequences cointroduced into the same cell will yield retroviral particles carrying defective retroviral genomes that carry therapeutic genes (e.g. Reviewed by Miller 1992). This cell is referred to as the producer cell (see below).

There are two common procedures for generating producer cells. In one, the sequences encoding retroviral Gag, Pol and Env proteins are introduced into the cell and stably integrated into the cell genome; a stable cell line is produced which is referred to as the packaging cell line. The packaging cell line produces the proteins required for packaging retroviral RNA but it cannot bring about encapsidation due to the lack of a psi region. However, when a vector genome (having a psi region) is introduced into the packaging cell line, the helper proteins can package the psi-positive recombinant vector RNA to produce the recombinant virus stock. This can be used to transduce the NOI into recipient cells. The recombinant virus whose genome lacks all genes required to make viral proteins can infect only once and cannot propagate. Hence, the NOI is introduced into the host cell genome without the generation of potentially harmful retrovirus. A summary of the available packaging lines is presented in “Retroviruses” (1997 Cold Spring Harbour Laboratory Press Eds: J M Coffin, S M Hughes, H E Varmus pp 449).

The present invention also provides a packaging cell line comprising a viral vector genome which is capable of producing a vector system of the invention. For example, the packaging cell line may be transduced with a viral vector system comprising the genome or transfected with a plasmid carrying a DNA construct capable of encoding the RNA genome. The present invention also provides a kit for producing a retroviral vector system of the invention which comprises a packaging cell and a retroviral vector genome.

The second approach is to introduce the three different DNA sequences that are required to produce a retroviral vector particle i.e. the env coding sequences, the gag-pol coding sequence and the defective retroviral genome containing one or more NOIs into the cell at the same time by transient transfection and the procedure is referred to as transient triple transfection (Landau & Littman 1992; Pear et al 1993). The triple transfection procedure has been optimised (Soneoka et al 1995; Finer et al 1994). WO 94/29438 describes the production of producer cells in vitro using this multiple DNA transient transfection method. WO 97/27310 describes a set of DNA sequences for creating retroviral producer cells either in vivo or in vitro for re-implantation.

The components of the viral system which are required to complement the vector genome may be present on one or more “producer plasmids” for transfecting into cells.

The present invention also provides a kit for producing a retroviral vector of the invention, comprising

(i) a viral vector genome which is incapable of encoding one or more proteins which are required to produce a vector particle;

(ii) one or more producer plasmid(s) capable of encoding the protein which is not encoded by (i); and optionally

(iii) a cell suitable for conversion into a producer cell.

In a preferred embodiment, the viral vector genome is incapable of encoding the proteins gag, pol and env. Preferably the kit comprises one or more producer plasmids encoding env, gag and pol, for example, one producer plasmid encoding env and one encoding gag-pol. Preferably the gag-pol sequence is codon optimised for use in the particular producer cell (see below).

The present invention also provides a producer cell expressing the vector genome and the producer plasmid(s) capable of producing a retroviral vector system of the present invention.

Preferably the retroviral vector system of the present invention is a self-inactivating (SIN) vector system.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it may be important to prevent the adventitious activation of an endogenous oncogene.

Preferably a recombinase assisted mechanism is used which facilitates the production of high titre regulated lentiviral vectors from the producer cells of the present invention.

As used herein, the term “recombinase assisted system” includes but is not limited to a system using the Cre recombinase/loxP recognition sites of bacteriophage P1 or the site-specific FLP recombinase of S. cerevisiae which catalyses recombination events between 34 bp FLP recognition targets (FRTs).

The site-specific FLP recombinase of S. cerevisiae which catalyses recombination events between 34 bp FLP recognition targets (FRTs) has been configured into DNA constructs in order to generate high level producer cell lines using recombinase-assisted recombination events (Karreman et al (1996) NAR 24:1616-1624). A similar system has been developed using the Cre recombinase/loxP recognition sites of bacteriophage P1 (see PCT/GB00/03837; Vanin et al (1997) J. Virol 71:7820-7826). This was configured into a lentiviral genome such that high titre lentiviral producer cell lines were generated.

By using producer/packaging cell lines, it is possible to propagate and isolate quantities of retroviral vector particles (e.g. to prepare suitable titres of the retroviral vector particles) for subsequent transduction of, for example, a site of interest (such as a DRG). Producer cell lines are usually better for large-scale production or vector particles.

Transient transfection has numerous advantages over the packaging cell method. In this regard, transient transfection avoids the longer time required to generate stable vector-producing cell lines and is used if the vector genome or retroviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al 1993, PNAS 90:8392-8396).

Producer cells/packaging cells can be of any suitable cell type. Producer cells are generally mammalian cells but can be, for example, insect cells.

As used herein, the term “producer cell” or “vector producing cell” refers to a cell which contains all the elements necessary for production of retroviral vector particles.

Preferably the envelope protein sequences, and nucleocapsid sequences are all stably integrated in the producer and/or packaging cell. However, one or more of these sequences could also exist in episomal form and gene expression could occur from the episome.

As used herein, the term “packaging cell” refers to a cell which contains those elements necessary for production of infectious recombinant virus which are lacking in the RNA genome. Typically, such packaging cells contain one or more producer plasmids which are capable of expressing viral structural proteins (such as gag-pol and env, which may be codon optimised) but they do not contain a packaging signal.

The term “packaging signal” which is referred to interchangeably as “packaging sequence” or “psi” is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon.

Packaging cell lines may be readily prepared (see also WO 92/05266), and utilised to create producer cell lines for the production of retroviral vector particles. As already mentioned, a summary of the available packaging lines is presented in “Retroviruses” (as above).

Also as discussed above, simple packaging cell lines, comprising a provirus in which the packaging signal has been deleted, have been found to lead to the rapid production of undesirable replication competent viruses through recombination. In order to improve safety, second generation cell lines have been produced wherein the 3′ LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection.

In these split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production. The packaging cell may be a cell cultured in vitro such as a tissue culture cell line. Suitable cell lines include but are not limited to mammalian cells such as murine fibroblast derived cell lines or human cell lines. Preferably the packaging cell line is a human cell line, such as for example: HEK293, 293-T, TE671, HT1080.

Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

It is highly desirable to use high-titre virus preparations in both experimental and practical applications. Techniques for increasing viral titre include using a psi plus packaging signal as discussed above and concentration of viral stocks.

As used herein, the term “high titre” means an effective amount of a retroviral vector or particle which is capable of transducing a target site such as a cell.

As used herein, the term “effective amount” means an amount of a regulated retroviral or lentiviral vector or vector particle which is sufficient to induce expression of the NOIs at a target site.

A high-titre viral preparation for a producer/packaging cell is usually of the order of 10⁵ to 10⁷ t.u. per ml. (The titer is expressed in transducing units per ml (t.u./ml) as titred on a standard D17 cell line). For transduction in tissues such as the DRG, it is necessary to use very small volumes, so the viral preparation is concentrated by ultracentrifugation. The resulting preparation should have at least 10⁸ t.u./ml, preferably from 10⁸ to 10⁹ t.u./ml, more preferably at least 10⁹ t.u./ml.

The presence of a sequence termed the central polypurine tract (cPPT) may improve the efficiency of gene delivery to non-dividing cells (see WO 00/31200). This cis-acting element is located, for example, in the EIAV polymerase coding region element. Preferably the genome of the vector system used in the present invention comprises a cPPT sequence.

In addition, or in the alternative, the viral genome may comprise a post-translational regulatory element and/or a translational enhancer.

Minimal Systems

It has been demonstrated that a primate lentivirus minimal system can be constructed which requires none of the HIV/SIV additional genes vif, vpr, vpx, vpu, tat, rev and nef for either vector production or for transduction of dividing and non-dividing cells. It has also been demonstrated that an EIAV minimal vector system can be constructed which does not require S2 for either vector production or for transduction of dividing and non-dividing cells. The deletion of additional genes is highly advantageous. Firstly, it permits vectors to be produced without the genes associated with disease in lentiviral (e.g. HIV) infections. in particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO-A-99/32646 and in WO-A-98/17815.

Thus, preferably, the delivery system used in the invention is devoid of at least tat and S2 (if it is an EIAV vector system), and possibly also vif, vpr, vpx, vpu and nef. More preferably, the systems of the present invention are also devoid of rev. Rev was previously thought to be essential in some retroviral genomes for efficient virus production. For example, in the case of HIV, it was thought that rev and RRE sequence should be included. However, it has been found that the requirement for rev and RRE can be reduced or eliminated by codon optimisation (see below) or by replacement with other functional equivalent systems such as the MPMV system. As expression of the codon optimised gag-pol is REV independent, RRE can be removed from the gag-pol expression cassette, thus removing any potential for recombination with any RRE contained on the vector genome.

In a preferred embodiment the viral genome of the first aspect of the invention lacks the Rev response element (RRE).

In a preferred embodiment, the system used in the present invention is based on a so-called “minimal” system in which some or all of the additional genes have been removed.

Codon Optimisation

Codon optimisation has previously been described in W099/41397. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimisation also overcomes the Rev/RRE requirement for export, rendering optimised sequences Rev independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with the exception of the sequence encompassing the frameshift site.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the gag-pol proteins.

For EIAV the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In a highly preferred embodiment, codon optimisation was based on lightly expressed mammalian genes. The third and sometimes the second and third base may be changed.

Due to the degenerate nature of the Genetic Code, it will be appreciated that numerous gag-pol sequences can be achieved by a skilled worker. Also there are many retroviral variants described which can be used as a starting point for generating a codon optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at in the HIV databases maintained by Los Alamos National Laboratory. Details of EIAV clones may be found in the NCBI database maintained by the U.S. National Institute of Health.

The strategy for codon optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev independent. In order to enable the use of anti-rev or RRE factors in the retroviral vector, however, it would be necessary to render the viral vector generation system totally Rev/RRE independent. Thus, the genome also needs to be modified. This is achieved by optimising vector genome components. Advantageously, these modifications also lead to the production of a safer system absent of all additional proteins both in the producer and in the transduced cell.

As described above, the packaging components for a retroviral vector include expression products of gag, pol and env genes. In addition, efficient packaging depends on a short sequence of 4 stem loops followed by a partial sequence from gag and env (the “packaging signal”). Thus, inclusion of a deleted gag sequence in the retroviral vector genome (in addition to the full gag sequence on the packaging construct) will optimise vector titre. To date efficient packaging has been reported to require from 255 to 360 nucleotides of gag in vectors that still retain env sequences, or about 40 nucleotides of gag in a particular combination of splice donor mutation, gag and env deletions. It has surprisingly been found that a deletion of all but the N-terminal 360 or so nucleotides in gag leads to an increase in vector titre. Thus, preferably, the retroviral vector genome includes a gag sequence which comprises one or more deletions, more preferably the gag sequence comprises about 360 nucleotides derivable from the N-terminus.

Pseudotyping

In the design of retroviral vector systems it is desirable to engineer particles with different target cell specificities to the native virus, to enable the delivery of genetic material to an expanded or altered range of cell types. One manner in which to achieve this is by engineering the virus envelope protein to alter its specificity. Another approach is to introduce a heterologous envelope protein into the vector particle to replace or add to the native envelope protein of the virus.

The term pseudotyping means incorporating at least a part of, or substituting a part of, or replacing all of, an env gene of a viral genome with a heterologous env gene, for example an env gene from another virus. Pseudotyping is not a new phenomenon and examples may be found in WO 99/61639, WO-A-98/05759, WO-A-98/05754, WO-A-97/17457, WO-A-96/09400, WO-A-91/00047 and Mebatsion et al 1997 Cell 90, 841-847.

Pseudotyping can improve retroviral vector stability and transduction efficiency. A pseudotype of murine leukemia virus packaged with lymphocytic choriomeningitis virus (LCMV) has been described (Miletic et al (1999) J. Virol. 73:6114-6116) and shown to be stable during ultracentrifugation and capable of infecting several cell lines from different species.

For pseudotyped vector systems of the present invention, the heterologous env region may be encoded by a gene which is present on a producer plasmid. The producer plasmid may be present as part of a kit for the production of retroviral vector particles suitable for use in the invention.

In the first preferred embodiment of the present invention, the vector system is administered directly to the DRG of a subject, for example by direct injection.

In this embodiment, if the vector system is a viral vector system, it may be pseudotyped with any heterologous env protein.

In the second preferred embodiment of the present invention, the vector system is administered to a site which is distant to the DRG. The vector system (or part thereof) then travels to the DRG by retrograde transport.

In this second embodiment, the vector system comprises an entity which enables it to travel by retrograde transport to the DRG. For example, the vector system may comprise a protein (or a mutant, variant, homologue or fragment thereof) from a virus which is capable of travelling by retrograde transport. For example, the system may comprise a protein from a rabies virus, herpes virus, adenovirus or from Ebola virus. If the vector system is a viral vector system, it may be pseudotyped with the envelope protein from such a virus. In a preferred embodiment, the vector system is pseudotyped with at least a part of a rabies G protein or a mutant, variant, homologue or fragment thereof.

In this case the vector system comprises a first nucleotide sequence coding for at least a part of an envelope protein; and one or more other nucleotide sequences derivable from a retrovirus that ensure transduction by the retroviral delivery system; wherein the first nucleotide sequence is heterologous with respect to at least one of the other nucleotide sequences; and wherein the first nucleotide sequence codes for at least a part of a rabies G protein or a mutant, variant, homologue or fragment thereof.

Rabies G Protein

In the present invention the vector system may be or comprise at least a part of a rabies G protein or a mutant, variant, homologue or fragment thereof. Where the vector system is a viral vector system, it may for example be pseudotyped with at least a part of a rabies G protein or a mutant, variant, homologue or fragment thereof.

Teachings on the rabies G protein, as well as mutants thereof, may be found in WO 99/61639 and well as Rose et al., 1982 J. Virol. 43: 361-364, Hanham et al., 1993 J. Virol.,67, 530-542, Tuffereau et al., 1998 J. Virol., 72,1085-1091, Kucera et al., 1985 J. Virol 55,158-162, Dietzschold et al., 1983 PNAS 80, 70-74, Seif et al, 1985 J.Virol., 53, 926-934, Coulon et al., 1998 J. Virol., 72, 273-278, Tuffereau et al., 1998 J. Virol., 72, 1085-10910, Burger et al., 1991 J.Gen. Virol. 72. 359-367, Gaudin et al 1995 J Virol 69, 5528-5534, Benmansour et al 1991 J Virol 65, 4198-4203, Luo et al 1998 Microbiol Immunol 42, 187-193, Coil 1997 Arch Virol 142, 2089-2097, Luo et al 1997 Virus Res 51, 35-41, Luo et al 1998 Microbiol Immunol 42, 187-193, Coll 1995 Arch Virol 140, 827-851, Tuchiya et al 1992 Virus Res 25, 1-13, Morimoto et al 1992 Virology 189, 203-216, Gaudin et al 1992 Virology 187, 627-632, Whitt et al 1991 Virology 185, 681-688, Dietzschold et al 1978 J Gen Virol 40 , 131-139, Dietzschold et al 1978 Dev Biol Stand 40, 45-55, Dietzschold et al 1977 J Virol 23, 286-293, and Otvos et al 1994 Biochim Biophys Acta 1224, 68-76. A rabies G protein is also described in EP-A-0445625.

The use of rabies G protein provides vectors which, in vivo, preferentially transduce targeted cells which rabies virus preferentially infects. This includes in particular neuronal target cells in vivo. For a neuron-targeted vector, rabies G from a pathogenic strain of rabies such as ERA may be particularly effective. On the other hand rabies G protein confers a wider target cell range in vitro including nearly all mammalian and avian cell types tested (Seganti et al., 1990 Arch Virol. 34,155-163; Fields et al, 1996 Fields Virology, Third Edition, vol.2, Lippincott-Raven Publishers, Philadelphia, New York).

The tropism of the pseudotyped vector particles may be modified by the use of a mutant rabies G which is modified in the extracellular domain. Rabies G protein has the advantage of being mutatable to restrict target cell range. The uptake of rabies virus by target cells in vivo is thought to be mediated by the acetylcholine receptor (AchR) but there may be other receptors to which it binds in vivo (Hanham et al, 1993 J. Virol.,67, 530-542; Tuffereau et al., 1998 J. Virol., 72, 1085-1091). It is thought that multiple receptors are used in the nervous system for viral entry, including NCAM (Thoulouze et al (1998) J. Virol 72(9):7181-90) and p75 Neurotrophin receptor (Tuffereau C et al (1998) Embo J 17(24) 7250-9).

The effects of mutations in antigenic site IlIl of the rabies G protein on virus tropism have been investigated and this region is not thought to be involved in the binding of the virus to the acetylcholine receptor (Kucera et al., 1985 J. Virol 55, 158-162; Dietzschold et al, 1983 Proc NatI Acad Sci 80, 70-74; Seif et al, 1985 J.Virol., 53, 926-934; Coulon et al., 1998 J. Virol., 72, 273-278; Tuffereau et al., 1998 J. Virol., 72, 1085-10910). For example a mutation of the arginine at amino acid 333 in the mature protein to glutamine can be used to restrict viral entry to olfactory and peripheral neurons in vivo while reducing propagation to the central nervous system. These viruses were able to penetrate motor neurons and sensory neurons as efficiently as the wild type virus, yet transneuronal transfer did not occur (Coulon et al., 1989, J. Virol. 63, 3550-3554). Viruses in which amino acid 330 has been mutated are further attenuated, being unable to infect either motor neurons or sensory neurons after intra-muscular injection (Coulon et al., 1998 J. Virol., 72, 273-278).

Alternatively or additionally, rabies G proteins from laboratory passaged strains of rabies may be used. These can be screened for alterations in tropism. Such strains include the following:

| Genbank accession number | Rabies Strain |
|---|---|
| J02293 | ERA |
| U52947 | COSRV |
| U27214 | NY 516 |
| U27215 | NY771 |
| U27216 | FLA125 |
| U52946 | SHBRV |
| M32751 | HEP-Flury |
| U17064 | Mokola G |
| * | |

Request ID for a blast of the sequence is available under 1000214283-16535-22519.

By way of example, the ERA strain is a pathogenic strain of rabies and the rabies G protein from this strain can be used for transduction of neuronal cells. The sequence of rabies G from the ERA strains is in the GenBank database (accession number J02293). This protein has a signal peptide of 19 amino acids and the mature protein begins at the lysine residue 20 amino acids from the translation initiation methionine. The HEP-Flury strain contains the mutation from arginine to glutamine at amino acid position 333 in the mature protein which correlates with reduced pathogenicity and which can be used to restrict the tropism of the viral envelope.

WO 99/61639 discloses the nucleic and amino acid sequences for a rabies virus strain ERA (Genbank locus RAVGPLS, accession M38452).

Mutants, Variants, Homologues and Fragments

The vector system of the second preferred embodiment of the present invention is or comprises at least part of a wild-type rabies G protein or a mutant, variant, homologue or fragment thereof.

The term “wild type” is used to mean a polypeptide having a primary amino acid sequence which is identical with the native protein (i.e., the viral protein).

The term “mutant” is used to mean a polypeptide having a primary amino acid sequence which differs from the wild-type sequence by one or more amino acid additions, substitutions or deletions. A mutant may arise naturally, or may be created artificially (for example by site-directed mutagenesis). Preferably the mutant has at least 90% sequence identity with the wild type sequence. Preferably the mutant has 20 mutations or less over the whole wild-type sequence. More preferably the mutant has 10 mutations or less, most preferably 5 mutations or less over the whole wild-type sequence.

The term “variant” is used to mean a naturally occurring polypeptide which differs from a wild-type sequence. A variant may be found within the same viral strain (i.e. if there is more than one isoform of the protein) or may be found within a different strains. Preferably the variant has at least 90% sequence identity with the wild type sequence. Preferably the variant has 20 mutations or less over the whole wild-type sequence. More preferably the variant has 10 mutations or less, most preferably 5 mutations or less over the whole wild-type sequence.

Here, the term “homologue” means an entity having a certain homology with the wild type amino acid sequence and the wild type nucleotide sequence. Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Besffit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Besffit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Besffit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O ), pyriylalanine, thienylalanine, naphthylalanine and phenylglycine.

Replacements may also be made by unnatural amino acids including; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-l-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The term “fragment” indicates that the polypeptide comprises a fraction of the wild-type amino acid sequence. It may comprise one or more large contiguous sections of sequence or a plurality of small sections. The polypeptide may also comprise other elements of sequence, for example, it may be a fusion protein with another protein. Preferably the polypeptide comprises at least 50%, more preferably at least 65%, most preferably at least 80% of the wild-type sequence.

The mutant, variant, homologue or fragment rabies G sequence should be capable of conferring the capacity for retrograde transport on the vector system.

The vector delivery system used in the present invention may comprise nucleotide sequences that can hybridise to the nucleotide sequence presented herein (including complementary sequences of those presented herein). In a preferred aspect, the present invention covers nucleotide sequences that can hybridise to the nucleotide sequence of the present invention under stringent conditions (e.g. 65° C. and 0.1 SSC) to the nucleotide sequence presented herein (including complementary sequences of those presented herein).

A potential advantage of using the rabies glycoprotein is the detailed knowledge of its toxicity to man and other animals due to the extensive use of rabies vaccines. In particular phase 1 clinical trials have been reported on the use of rabies glycoprotein expressed from a canarypox recombinant virus as a human vaccine (Fries et al., 1996 Vaccine 14, 428-434), these studies concluded that the vaccine was safe for use in humans.

EOIs/NOIs

In a broad aspect, the present invention relates to a vector system that is capable of transporting an entity of interest (“EOI”).

The EOI may be a chemical compound, a biological compound or combinations thereof. By way of example, the EOI may be a protein (such as a growth factor), a nucleotide sequence, an organic and/or an inorganic pharmaceutical (such as an analgesic, an anti-inflammatory, a hormone, a lipid), or combinations thereof.

Preferably the EOI is one or more NOIs (nucleotide sequences of interest).

If the vector system of the present invention is a viral vector system, then it is possible to manipulate the viral genome so that viral genes are replaced or supplemented with one or more NOIs which may be heterologous NOIs.

The term “heterologous” refers to a nucleic acid or protein sequence linked to a nucleic acid or protein sequence to which it is not naturally linked.

In the present invention, the term NOI includes any suitable nucleotide sequence, which need not necessarily be a complete naturally occurring DNA or RNA sequence. Thus, the NOI can be, for example, a synthetic RNA/DNA sequence, a recombinant RNA/DNA sequence (i.e. prepared by use of recombinant DNA techniques), a cDNA sequence or a partial genomic DNA sequence, including combinations thereof. The sequence need not be a coding region. If it is a coding region, it need not be an entire coding region. In addition, the RNA/DNA sequence can be in a sense orientation or in an anti-sense orientation. Preferably, it is in a sense orientation. Preferably, the sequence is, comprises, or is transcribed from cDNA.

The retroviral vector genome may generally comprise LTRs at the 5′ and 3′ ends, suitable insertion sites for inserting one or more NOI(s), and/or a packaging signal to enable the genome to be packaged into a vector particle in a producer cell. There may even be suitable primer binding sites and integration sites to allow reverse transcription of the vector RNA to DNA, and integration of the proviral DNA into the target cell genome. In a preferred embodiment, the retroviral vector particle has a reverse transcription system (compatible reverse transcription and primer binding sites) and an integration system (compatible integrase and integration sites).

The NOIs may be operatively linked to one or more promoter/enhancer elements. Transcription of one or more NOI may be under the control of viral LTRs or alternatively promoter-enhancer elements can be engineered in with the transgene. Preferably the promoter is a strong promoter such as CMV. The promoter may be a regulated promoter. The promoter may be tissue-specific or cell-specific. In a preferred embodiment the promoter is neuron-specific. Especially preferred are promoters which restrict expression to C and/or Aδ fibres. In this way it is possible to avoid gene expression in the larger myelinated fibres which are responsible for transmission of other sensory stimuli

Expression of the NOI may be inducible. Transcription of the NOI may thus be controlled, for example to modulate effective analgesia in pain applications. Inducible promoters include those regulated by hormones and hormone analogs such as progesterone, ecdysone and glucocorticoids as well as promoters which are regulated by tetracycline, heat shock, heavy metal ions, and lactose operon activating compounds.

The EOI/NOI may be or encode a protein of interest (“POI”). In this way, the vector delivery system could be used to examine the effect of expression of a foreign gene on the target cell. For example, the retroviral delivery system could be used to screen a cDNA library for a particular effect on the sensory neuron (or an alternative target cell).

The EOI/NOI may encode or be a cytoplasmic protein, nuclear protein, membrane protein, or secreted protein.

The EOI/NOI may be capable of integrating in the genome of a target cell.

The EOI/NOI may be capable of blocking or inhibiting the expression of a gene in the target cell. For example, the NOI may be an antisense sequence or an siRNA. The inhibition of gene expression using antisense technology is well known.

In one embodiment, the NOI comprises an siRNA. Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi). The mechanism of RNAi involves the processing of long dsRNAs into duplexes of 21-25 nucleotide (nt) RNAs. These products are called small interfering or silencing RNAs (siRNAs) which are the sequence-specific mediators of mRNA degradation. In differentiated mammalian cells dsRNA >30 bp has been found to activate the interferon response leading to shut-down of protein synthesis and non-specific mRNA degradation (Stark et al 1998). However this response can be bypassed by using 21 nt siRNA duplexes (Elbashir et al 2001, Hutvagner et al 2001) allowing gene function to be analysed in cultured mammalian cells.

The EOI/NOI or a sequence derived from the NOI may be capable of “knocking out” the expression of a particular gene in the target cell. There are several “knock out” strategies known in the art. For example, the NOI may be capable of integrating in the genome of the target cell so as to disrupt expression of the particular gene. The NOI may disrupt expression by, for example, introducing a premature stop codon, by rendering the downstream coding sequence out of frame, or by affecting the capacity of the encoded protein to fold (thereby affecting its function).

Alternatively, the EOI/NOI may be capable of enhancing or inducing ectopic expression of a gene in the target cell. The NOI or a sequence derived therefrom may be capable of “knocking in” the expression of a particular gene.

The EOI may have or encode a protein which has a therapeutic effect. For example, an NOI delivered by the vector delivery system may be a therapeutic gene—in the sense that the gene itself may be capable of eliciting a therapeutic effect or it may code for a product that is capable of eliciting a therapeutic effect.

In accordance with the present invention, suitable EOIs include those that are (or can produce entities) of therapeutic and/or diagnostic application such as, but not limited to: cytokines, chemokines, hormones, antibodies, anti-oxidant molecules, engineered immunoglobulin-like molecules, a single chain antibody, fusion proteins, enzymes, immune co-stimulatory molecules, immunomodulatory molecules, anti-sense RNA, siRNA, a transdominant negative mutant of a target protein, a toxin, painal toxin, an antigen, a tumour suppresser protein and growth factors, vasoactive proteins and peptides, anti-viral proteins, ribozymes, receptor proteins and ion channels and derivatives thereof. The EOI may be an NOI which encodes a member of this list.

The EOI/NOI may also be or encode an antiapoptotic factor or a neuroprotective molecule. The survival of cells during programmed cell death depends critically on their ability to access “trophic” molecular signals derived primarily from interactions with other cells. For example, the NOI may encode a neurotrophic factor, such as ciliary neurotrophic factor (CNTF) or glial cell-derived neurotrophic factor (GDNF) or it may be a gene involved in control of the cell death cascade (such as Bcl-2). This may be useful in therapeutic strategies involving arresting neuronal and glial cell death induced by injury, disease, and/or aging in humans.

Neurotrophic factors are proteins which promote the survival of specific neuronal populations. Many have other physiological effects on neurons as inducing morphological differentiation, enhancing nerve regeneration, and stimulating neurotransmitter expression. These properties suggest that neurotrophic factors are highly promising as potential therapeutic agents for neurological diseases. Glial cell line-derived neurotrophic factor (GDNF) will most likely be applied to peripheral sensory neurons, since GFPα3 (one of the GDNF receptors) receptor is expressed predominantly in nociceptive sensory neurons. It has been demonstrated recently that GDNF both prevents and reverses sensory abnormalities that developed in neuropathic pain models. This effect is thought to occur as a consequence of the reversal by GDNF of the injury-induced plasticity of several sodium channel subunits.

In a preferred embodiment, the EOI may be capable of biasing the transcriptome and/or proteotome of a cell. For example, the EOI may be one which is known to modulate gene expression in a given cell (such as a neuron). CREB (cyclic AMP element binding protein) is one such protein (Kornhauser et al., (2002) Neuron 34:221-233). DREAM is thought to act as a suppressor of transcription, for example of endogenous opiates (Cheng et al., 2002. Cell 108:31-43). Alternatively the EOI may act further upstream in the pathway. For example, the EOI may modulate the activity of a second entity which has the capacity to modulate gene expression. ERK MAP kinase has the capacity to activate CREB, and as such is an example of an “upstream factor” (Ji et al., (2002). J. Neurosci. 22:478-85).

The present invention also relates to a method of screening EOIs for their potential in preventing and/or treating and/or relieving pain. A test EOI may be screened individually, or a plurality of test EOIs may be screened simultaneously or sequentially. In a preferred embodiment the screening method is wholly or partly automated, facilitating the screening of a large number of test compounds.

For example, libraries of test compounds may be screened in multi-well plates (e.g., 96-well plates), with a different test compound in each well. In particular, the library of candidate compounds may be a combinatorial library. A variety of combinatorial libraries of random-sequence oligonucleotides, polypeptides, or synthetic oligomers have been proposed and number of small-molecule libraries have also been developed. Combinatorial libraries of oligomers may be formed by a variety of solution-phase or solid-phase methods in which mixtures of different subunits are added stepwise to growing oligomers or parent compound, until a desired oligomer size is reached (typically hexapeptide or heptapeptide). A library of increasing complexity can be formed in this manner, for example, by pooling multiple choices of reagents with each additional subunit step. Alternatively, the library may be formed by solid-phase synthetic methods in which beads containing different-sequence oligomers that form the library are alternately mixed and separated, with one of a selected number of subunits being added to each group of separated beads at each step. Libraries, including combinatorial libraries are commercially available form pharmaceutical companies and speciality library suppliers.

In a preferred embodiment the library of EOIs is a cDNA library. cDNA libraries are commercially available or may be generated in the laboratory (for example from total cellular mRNA).

Cellular Excitability and Ion Channels

In a highly preferred embodiment the EOI is capable of modulating the cellular excitability of a target cell. For example, the EOI may be capable of causing hyperpolarisation of the target cell.

As mentioned above, ion channels in the membrane of cells can open, allowing specific ions to diffuse across the plasma membrane down their electrochemical gradient, to modulate the membrane potential of an excitable cell. Examples of such ion channels include: potassium channels, sodium channels, calcium channels and chloride channels.

The permeability of the cell membrane to potassium ions is particularly important. The resting membrane potential of a cell is an approximation of the potassium equilibrium potential. It is fundamentally dependent upon the permeability of the cell membrane to potassium ions. Over-activity of potassium channels leads to cell hyperpolarization and potassium channel openers (minoxidil) are in current therapeutic use, for example to cause a decrease in vascular tone. Activation of sodium or calcium channels and in some cases chloride channels, on the other hand, results in a net inward current (movement of net positive charge) and cell depolarisation.

Preferably the EOI is capable of modulating the expression and/or activity of an ion channel. An ion channel is a protein which allows an ionic current to flow across a membrane. For example, the EOI may be capable of modulating the expression of a potassium channel, a sodium channel, a calcium channel, a chloride channel and/or a non-selective cation channel.

In a highly preferred embodiment, the EOI is capable of causing the expression of a potassium channel or part thereof. The potassium channel may be constitutively active.

Examples of species that may be used to modulate membrane potential include (but are not limited to):

1. Ion channel subunits (native)

2. Ion channel subunits (mutant—see below)

3. Peptide activators of ion channels, for example βγ subunits of the G protein family have been shown to activate potassium channels in the heart.

4. Peptide blockers of ion channels

5. Antisense sequences or siRNAs which are capable of inhibiting the expression of ion channels

Mutant ion channels or subunit thereof may be constitutively active. Alternatively, they may be modified to include a gate which is controlled by an endogenous, or more preferably an exogenous signal (such as a drug-induced activator). The mutant channel or subunit may be modified to remove the control by an endogenous signal, for example by deleting the site responsible for control by PKC, PKA or pH.

Voltage-Gated Sodium Channels

Voltage-gated sodium channels play a central role in the initiation of action potentials in all neurons, therefore they represent a major target in the prevention of this hyperexcitability. The increase of excitability or variation of the baseline sensitivity in the primary sensory neurons leads to abnormal signal generation along the nociceptive pathway. In addition, several pharmacological approaches have demonstrated that specific sodium channel blockers, such as anti-convulsants, anti-depressants and local anaesthetics may be effective for the treatment of pain.

Multiple distinct sodium channels encoded by different genes (TypeI, II, III, SM1, SM2, Na6, PN1, SNS2) are present in DRG. Some of these sodium channels are sensory neuron-specific and expressed in specific subpopulations of nociceptors (PN1, SNS, NaG). Two types of sodium channel have been differentiated on the basis of their kinetic and sensitivity to the neurotoxin tetrodotoxin (TTX). The fast-inactivating TTX-sensitive currents, are found in all DRG cells. The slowly inactivating TTX-resistant currents appear to be preferentially expressed in specific subpopulation of sensory neurons and are sensory neuron specific. Sodium channel expression within DRG neurons changes both during development, and also in various pain states. A loss of TTX-resistant currents has been described after axonal transection and expression of SNS is down-regulated. TTX-resistant sodium currents are however upregulated during inflammation.

Sodium channels have marked voltage sensitivity, and are composed of a principal alpha subunit and one or more smaller beta subunits. Only a single alpha subunit is required for channel activity and the subunit is four times as big as a 6tm Kv channel (equivalent to all four subunits being in one gene). There are c.10 genes, SCN1A, SCN2A, SCN3A, SCN4A, SCN5A, SCN6A, SCN7A, SCN8A, SCN1B1, SCN1B2. The beta subunit only has one single tm domain and modifies the alpha subunit current.

In a preferred embodiment the EOI is capable of modulating the expression or activity of voltage-gated sodium channels.

Potassium Channels

Potassium channels fall into two main structural families: those having 6 or 2 transmembrane domains. There are six conserved families with 6 tm regions including voltage gated, KCNQ, and eag-like channels as well as three families of Ca-activated channels. The channels of the three named families are generally closed at the resting potential but open at depolarized potentials and are involved in events such as repolarization.

6tm Families

| | |
|---|---|
| Kv: | Shaker (Kv1), Shab (Kv2), Shaw (Kv3), |
| | Shal (Kv4) and, Shaker divided into |
| | Kv1.1-Kv1.9. |
| KCNQ: | KCNQ1, 2, 3 |
| Eag: | eag, erg, elk |
| BK: | Slo, nSlo2 |
| SK: | SK1, 2, 3 |
| IK | |

The voltage gated K channels are made up of pore forming alpha subunits which may be associated with one of a number of beta subunits. Four alpha subunits are required for a functional channel. The beta subunits are cytoplasmic and modify alpha subunit expression and channel activity.

In the Shaker alpha subunit, at the distal end of the N terminus, there are a number of residues that act as an inactivation ball by plugging the pore.

KIR Channels

These channels stabilize the resting membrane potential near the K equilibrium potential and show strong inward rectification (pass less outward current in response to a hyperpolarizing potential step than inward current in response to a depolarizing step of the same amplitude. There are six main subfamilies, and there are a number of different isoforms and splice variants for each gene. As with the Kv channels, KIR can form heteromultimers which affects currents.

Kir: 1.1(ROMK1), 4.1, 6.1, 6.2(KATP), 2.1(IRK), 2.2, 2.3, 3.2(GIRK2), 3.4(GIRK4), 3.3(GIRK3), 3.1(GIRK1), 5.1 (old names given in parentheses)

KIR 6.1 and 6.2 do not form functional channels without the sulphonyl urea receptor (SUR1 and SUR2).

In a preferred embodiment the EOI is capable of modulating the expression or activity of a potassium channel.

Calcium Channels

The six types of calcium channel, T, L, N, P, Q, R and are distinguished by their sensitivity to pharmacological blockers. They are composed of many different subunits which coassemble. For example, in skeletal muscle the L-type channels are made up from alphal, 2, beta, gamma and delta subunits in a 1.1.1.1.1 stoichiometry.

In a preferred embodiment the EOI is capable of modulating the expression or activity of a calcium channel.

ASICs

Acid-sensing ion channels (ASICs) are members of the amiloride sensitive sodium channel/ENaC family of ion channels. They are expressed in the central nervous system and in sensory neurons and are activated by extracellular protons. Painful conditions such as ischaemia and inflammatory disorders are associated with tissue acidosis (pH7 - pH5) with the sensation being a result of the direct action of protons on ASICs.

Given the number of different insults that may contribute to the initiation of the painful stimulus other than those associated with tissue acidosis, it is more effective to inhibit the transmission of the action potential (e.g. at the level of the DRG) rather than to inhibit a potential component of the initial signal generation. In this sense, ASICs merely act as the receptors for the initiation of the action potential and are not specific targets. In contrast, preferred targets for the present invention are those that allow general inhibition of cellular excitability or of propagation of the action potential and as such will decrease the transmission of any number of painful stimuli. Accordingly, in preferred embodiments of the invention, the EOI is capable of modulating the expression or activity of a voltage -gated sodium channel, a potassium channel or a calcium channel and not an ASIC.

Receptors

Alternatively, the EOI may be capable of modulating the expression or activity of a receptor, in particular a receptor found on cells which are located wholly or partly within the DRG.

Opioids, receptors for which are located on peripheral and central neurons, are the treatment of choice during acute post-operative pain. In nerve injury, the down-regulation of opioid receptors, especially μ-receptors in DRG could explain the ineffectiveness of opioids treatment in neuropathic pain. More recent studies suggest that a specific population of the rostroventromedial medulla (RVM) neurons, those expressing opioid μ-receptors, is critical in the behavioural expression of experimental animals. Neurons in the RVM project to the spinal cord loci where the neurons inhibit or facilitate pain transmission. Inhibition of neuropathic pain has been shown by selective ablation of RVM cells expressing the μ-opioid receptor in spinal nerve ligation injury model. This result suggests that the descending projections from the brainstem have an important role in facilitating pain transmission.

In order to treat pain, the vector system of the present invention may be used to deliver an EOI which is capable of inhibiting expression of μ-receptors, particularly in RVM neurons in the brain. For example, the EOI may be or encode an antisense sequence for μ-receptors. Conversely the EOI may be capable of causing the overexpression of opioid receptors for the treatment of conditions in which opioid receptors are down-regulated. If the number of opioid receptors in the DRG was increased, this may enhance the effectiveness of pain relief by administration of opioids.

Pain hypersensitivity is largely an expression of changes,in the excitability of neurons of the spinal cord with alteration of the properties of the NMDA receptors. NMDA antagonists, such as ketamine or dextrometorphan have been demonstrated to be effective in neuropathic pain treatment. If NMDA receptors are involved in pain, it should be possible to control such pain if the EOI is or encodes an entity which blocks the expression or activity of such receptors.

The EOI may be capable of modulating the activity or expression of an antinociceptive target, such as an NK1 receptor, PCK-γ, VR1 receptor, NMDA receptor or an N-type calcium channel. For example, the EOI may encode antisense or si RNA sequences against these receptors.

The EOI may be capable of modulating the activity or expression of a pronociceptive target, such as a CB_{1/2} receptor, PCK-γ, mACH receptor, nACH receptor, opioid receptor or an α2 adrenergic receptor.

Target Cell

The EOI is capable (directly or indirectly) of exerting an effect on a target cell. If the EOI is delivered to a sensory neuron within the DRG, this may be the target cell. Alternatively, the target cell may be a different cell. For example, following delivery to the sensory neuron, the EOI (in the same or a different form i.e. optionally modified) may move on to a different target cell.

For example, the EOI may be an NOI which is capable of encoding a secretable protein. Once secreted, the protein may exert an effect on a target cell (for example a neighbouring cell). For some applications, the NOI expression product may demonstrate a general bystander effect; that is the production of the expression product in one cell leading to the modulation of additional, related cells, either neighbouring or distant, which possess a common phenotype.

The target cell may be a sensory neuron. Alternatively the target cell may be a different cell type. The target cell may be a cell found in the DRG, such as a glial cell. Preferably the target cell neighbours the cell body of the sensory neuron which receives the EOI. Alternatively, the target cell may be a cell found in the peripheral nervous tissue, such as a neuron, a glial cell or a Schwann cell. The present invention also provides a method of delivering an (optionally modified) EOI to the spinal cord, in which case the target cell may be, for example, a motorneuron, an interneuron, a glial cell, an astrocyte or an oligodendrocyte.

Preferably the target cell is a cell within or which passes through the DRG. Apart from sensory neurons (the cell bodies for which are within the DRG), the DRG also comprises glial cells.

Preferably the target cell is a sensory neuron. Especially preferred is a sensory neuron either within a C fiber or an Aδ fiber. C fiber neurons are especially preferred.

Sensory neurons are pseudo-unipolar neurons having a single process which projects from the cell body. This process bifurcates to form terminals in the periphery (the peripheral branch) and in the grey matter of the spinal cord where they synapse with other neurons (the central branch).

If the EOI is delivered to the cell body of a sensory neuron in the DRG it can then travel (with or without modification) to the spinal cord via the central branch. Once in the spinal cord the EOI or derivative (which may be a modified EOI) thereof can then exert an effect on other cells, such a motor neurons, or interneurons.

The present invention also provides, therefore, a method for delivering an EOI to the spinal cord, which comprises the following steps:

(i) delivery of an EOI to the cell body of a sensory neuron using a vector system of the second preferred embodiment of the present invention;

(ii) optional modification of the EOI; and

(iii) delivery of the optionally modified EOI from the cell body of the sensory neuron to the spinal cord via the central branch of the sensory neuron.

The EOI may thus be modified by any suitable means. The nature of the modification will of course depend on the nature of the EOI but includes any alteration of the EOI which occurs in the cell body, including translation of the NOI to a POI (which may thus be the modified EOI), and processing steps in the Golgi apparatus, such as post-translational modification, glycosylation reactions etc.

For example, the vector system may deliver an NOI to the cell body of a sensory neuron. The NOI may be translated into a POI within the cell body and the POI delivered to the spinal cord via the central branch.

In this embodiment, the (optionally modified) EOI may, for example, be capable of modulating the activity or expression of a neurotransmitter, a neurotrophin (such as GDNF, BDNF, NT3, CTNF and nerve growth factor), an antiapoptotic factor, an ion channel and or a receptor.

This method may be used for non-invasive access to the CNS, and so it is suitable for the treatment and/or prevention of any condition which affects the brain and/or spinal cord. These include conditions associated with motor neurons, such as motor neuron disease. For example, Amyotrophic Lateral Sclerosis (ALS) may be treatable with the use of anti-apoptotic factors. Spinal muscular atrophy (in neonates) may be preventable or treatable by replacing survival motor neuron gene 1, in order to avoid apoptosis. These also include other conditions associated with sensory neurons. For example encephalins may be used to regrow sensory neurons in conditions such as paraplegia.

The present invention also relates to drug discovery and validation methods, where the effect of a test EOI on a particular target cell is monitored. In this aspect, the target cell may be in vivo or in vitro. The target cell may be any cell type which has the capacity to exhibit a monitorable change in response to a test EOI. The monitorable change should indicate the potential relevance of the EOI in the prevention and/or treatment of pain.

Preferably the target cell is in situ within a DRG or is derivable from a DRG in vitro.

DRG

Spinal cord organisation appears to be segmented because the 31 pairs of spinal nerves emerge at regular intervals. Spinal nerves are the paths of communication between the spinal cord and the nerves innervating specific regions of the body. Two bundles of axons, called “roots”, connect each spinal nerve to a segment of the cord. The dorsal root contains sensory fibres, which conduct impulses from the periphery to the central nervous system. Each dorsal root has a swelling, the dorsal root ganglion (DRG) which contains the cell bodies of sensory neurons. The ventral root contains axons of motor neurons, which conduct impulses from the CNS to effector organs and cells.

Sensory neurons originate in the DRG. Different DRGs provide a somatosensory representation of the body. The area of skin innervated by a single dorsal root is called a dermatome. Topographical maps are available of the DRGs within the human body.

By administration of the vector system such that it delivers an EOI to a DRG of the subject, the EOI is delivered to a particular subset of sensory neurons.

The present invention also provides a method for identifying and/or validating an EOI with potential for pain relief. In the method, the effect of a test EOI on a target cell is monitored. In a preferred aspect, the target cell is a cell of the DRG in situ or in culture. In vitro DRG-derived cell cultures include dissociated or explant cultures. Methods for producing such cultures are known in the art (see for example Voilley et al., (2001) J.Neurosci. 21:8026-33; Gilabert and McNaughton (1997) J Neurosci Methods 71:191-8).

Pain

The vector system of the present invention may be suitable for use in a method for treating and/or preventing pain.

Pain can be classified into two major types, fast and slow. Fast pain has been described as sharp, acute, pricking or electric while slow pain has been referred to as chronic, burning, aching, throbbing or nauseous. The nerve fibres that are responsible for pain transmission are the A* and C fibres. Fast pain is carried by A* fibres with conduction velocities of 6-30 m.s⁻¹. These fibres are activated by mechanical or thermal stimuli. By contrast, C fibres are activated either by chemical (slow-chronic) pain or by persistent mechanical/thermal stimuli and have conduction velocities of 0.5-2 m.s⁻¹.

Transmission of pain from the periphery into the central nervous system occurs through dual pathways—in the neospinothalamic and paleospinothalamic tracts. The neospinothalamic tract is predominantly formed by the A* fibres which terminate mainly in the lamina marginalis of the dorsal horn. Pain transmission in the paleothalamic tract is carried mainly, but not exclusively, by C fibres. These fibres terminate in the substantia gelatinosa. Both tracts pass through the anterior commissure and then pass upward to the brain in the anterolateral pathway before terminating in the reticular nuclei of the brainstem or thalamus.

The vector system of the present invention is particularly suitable for the treatment of chronic intransient pain. Examples of such pain is that associated with conditions such as cancer, osteo and rheumatoid arthritis, back pain, sciatica and multiple sclerosis. The system is also useful for treating post-operative pain.

In a particularly preferred embodiment the vector system of the present invention is used for the treatment of neuropathic pain, a maladaptive form of pain which occurs after peripheral or central nervous system injury. Neuropathic pain is initiated or caused by a primary lesion or dysfunction of the nervous system. It includes diabetic neuropathy, cancer-related and HIV-related pain.

Administration Routes

The vector system of the present invention is administered such that the EOI is delivered to a DRG of the subject.

In a first preferred embodiment of the present invention, the vector system is administered directly to the DRG. Preferably the vector system is injected directly into the DRG.

In applications where the administration site and the target site are different, problems can arise from unwanted delivery of the EOI to cells surrounding the administration site. Direct administration (such as injection) has the advantage that, since the administration site is the same as the target site, there can be no side effects associated with “bleeding” from the administration site.

Direct administration by injection to the DRG also has the advantage that, by choosing a particular DRG, a particular sub-set of sensory neurons will be locally targetted. Topographical maps of the DRGs have been prepared, with the area of skin innervated by a single dorsal root called a dermatome.

In a second preferred embodiment the vector system is administered to a site which is distant to the DRG but at least part of the system travels to the DRG by retrograde transport.

Administration to a site which is distant to the DRG is advantageous because access to the distal site may be easier than access to the DRG. Also, by using retrograde transport is it possible to deliver the EOI to certain cells or groups of cells. For example, where the vector system is administered peripherally at the site of pain, the vector system (or part thereof) will travel to the DRG by retrograde transport and deliver the EOI to cells which are directly involved in sensing the pain.

There are other administration sites which may be used for this embodiment of the invention which are easier to access than the DRG, such as the dorsal horn of the spinal cord, or the sciatic nerve. Injection into the DRG or sciatic nerve may be used to increase transduction in the DRG relative to a more distant site (injection into a footpad or the site of pain).

In this embodiment the vector system comprises an entity which renders it capable of travelling by retrograde transport. For example the vector system may comprise one or more features from a virus (such as polio virus, rabies virus, HSV or adenovirus) which are capable of retrograde transport in vivo. The vector system may comprise at least part of an envelope protein from such a virus or a mutant, variant, homologue or fragment thereof.

Preferably the vector system is or comprises at least part of a rabies G protein or a mutant, variant, homologue or fragment thereof (see above).

Retrograde Transport

The cell body is where a neuron synthesises new cell products. Two types of transport systems carry materials from the cell body to the axon terminals and back. The slower system, which moves materials 1-5 mm per day is called slow axonal transport. It conveys axoplasm in one direction only (from the cell body toward the axon terminals (anterograde transport)). There is also “Fast transport” which is responsible for the movement of membranous organelles at 50-200 mm per day away from the cell body (anterograde) or back to the cell body (retrograde) (Hirokawa (1997) Curr Opin Neurobiol 7(5):605-614).

Vector systems comprising rabies G protein are capable of retrograde transport (i.e. travelling towards the cell body). The precise mechanism of retrograde transport is unknown, however. It is thought to involve transport of the whole viral particle, possibly in association with an internalised receptor. The fact that vector systems comprising rabies G can be specifically transported in this manner (as demonstrated herein) suggests that the env protein may be involved.

HSV, adenovirus and hybrid HSV/adeno-associated virus vectors have all been shown to be transported in a retrograde manner in the brain (Horellou and Mallet (1997) Mol Neurobiol 15(2) 241-256; Ridoux et al (1994) Brain Res 648:171-175; Constantini et al (1999) Human Gene Therapy 10:2481-2494). Injection of Adenoviral vector system expressing glial cell line derived neurotrophic factor (GDNF) into rat striatum allows expression in both dopaminergic axon terminals and cell bodies via retrograde transport (Horellou and Mallet (1997) as above; Bilang-Bleuel et al (1997) Proc. Natl. Acd. Sci. USA 94:8818-8823).

Retrograde transport can be detected by a number of mechanisms known in the art. For example, it is known to monitor labelled proteins or viruses and directly monitor their retrograde movement using real time confocal microscopy (Hirokawa (1997) as above).

In the present invention, the vector system (or part thereof) is capable of travelling from the administration site to the DRG by retrograde transport. In a preferred embodiment, the vector system travels up the axon of a sensory neuron, to the cell body (within the DRG).

In a preferred embodiment the vector system is administered to a peripheral administration site. The vector may be administered to any part of the body from which it can travel to the DRG by retrograde transport. In other words the vector may be administered to any part of the body to which a sensory neuron projects.

The “periphery” can be considered to be all part of the body other than the CNS (brain and spinal cord). In particular, peripheral sites are those which are distant to the CNS. Sensory neurons may be accessed by administration to any tissue which is innervated by the neuron. In particular this includes the skin, muscles and the sciatic nerve.

An advantage with the peripheral administration system is that it is possible to target particular groups of cells (e.g. sets of neurons), or a particular neural tract by choosing a particular administration site. Where a subject is suffering from pain (in particular slow, chronic pain), the particular sensory neuron(s) involved in transmitting the pain may be targeted by administration of the vector system directly into the area of pain.

Screening Methods

The present invention provides a method for identifying and/or validating new drugs for use in pain therapy.

For example, there is provided a screening method for identifying new EOI(s) which are useful in the prevention and/or treatment of pain.

There is provided a method for identification and/or validation of an EOI useful in the prevention and/or treatment of pain which comprises the step of

(i) delivery of a test EOI to target cell;

(ii) analysis of the effect of the EOI on the target cell; and

(iii) selection of an EOI with therapeutic potential.

The term “test EOI” is used to indicate a candidate EOI whose usefulness in the prevention and/or treatment of pain is under investigation. The test EOI may be a single entity or it may be one of a plurality of compounds being tested either simultaneously or sequentially. Preferably a large number of EOIs may be screened using the method and those which show potential are selected. This type of large-scale screening method may conveniently be automated.

As mentioned above, the target cell may be in vivo or in vitro. The type of analysis suitable to screen the EOI will depend on the predicted effect of the EOI and on the nature and location of the target cell.

In a preferred embodiment, the EOI affects transcription/translation of one or more genes in the target cell. For example, the EOI may be an antisense sequence which binds the nascent transcript, which may cause its degradation (e.g. by RNAse H) and/or block its translation. In another embodiment, the EOI may be an siRNA. Transcription and/or translation of a given gene may be detected by a number of methods known in the art. The presence or absence of a particular RNA may be detected, for example, by RT-PCT, Northern blotting or In situ hybridisation. The protein encoded by the gene may be detected by Western blotting or, if an antibody specific for the protein is available, ELISA or FACS analysis.

The EOI may affect expression of one or more genes. The pool of RNAs expressed in a cell is sometimes referred to as the transcriptome. Methods for measuring the transcriptome, or some part of it, are known in the art. A collection of articles summarizing some current methods appeared as a supplement to the journal Nature Genetics. (The Chipping Forecast. Nature Genetics supplement, volume 21, January 1999.) A preferred method for measuring expression levels of mRNAs is to spot PCR products corresponding to a large number of specific genes on a nylon membrane such as Hybond N Plus (Amersham-Pharmacia). Total cellular mRNA is then isolated, labeled by random oligonucleotide priming in the presence of a detectable label (e.g. alpha 33P labeled radionucleotides or dye labeled nucleotides), and hybridized with the filter containing the PCR products. The resulting signals can be analyzed by commercially available software, such as can be obtained from Clontech/Molecular Dynamics or Research Genetics, Inc.

Experiments have been described in model systems that demonstrate the utility of measuring changes in the transcriptome before and after changing the growth conditions of cells, for example by changing the nutrient environment. The changes in gene expression help reveal the network of genes that mediate physiological responses to the altered growth condition. Similarly, the addition of a drug to the cellular or in vivo environment, followed by monitoring the changes in gene expression can aid in identification of gene networks that mediate physiological and pharmacological responses. A similar approach could be used to screen EOIs which, for example, act as growth factors.

EOIs which bias the transcriptome in neurons are known in the art. For example, increased intracellular calcium concentrations or ERK MAP kinase are thought to activate CREB (cyclic AMP element binding protein) to modulate gene expression. (Kornhauser et al., (2002) Neuron 34:221-233; Ji et al., (2002) J. Neurosci. 22:478-85). Also, DREAM is thought to act as a suppressor of transcription of endogenous opiates. (Cheng et al., (2002). Cell 108:31-43). The EOI of the present invention may have a similar capacity to bias the transcriptome or it may a gene whose expression is controlled (i.e. repressed or activated) by such an EOI. Either may be detected using a screening method of the present invention.

Thus, novel genes may be identified as a result of their capacity to modulate the transcriptome/proteotome or as a result of EOI-induced modulation of their transcription/translation. For example, a gene could be identified by expressing ERK MAP kinase, CREB or DREAM (possibly in a constitutively active form) and examining the resulting change in gene expression.

The pool of proteins expressed in a cell is sometimes referred to as the proteome. Studies of the proteome may include not only protein abundance but also protein subcellular localization and protein-protein interaction. Methods for measuring the proteome, or some part of it, are known in the art. One widely used method is to extract total cellular protein and separate it in two dimensions, for example first by size and then by isoelectric point. The resulting protein spots can be stained and quantitated, and individual spots can be excised and analyzed by mass spectrometry to provide definitive identification. The results can be compared from two target cell samples, only one of which has been treated with the EOI.

The differential up or down modulation of specific proteins in response to EOI treatment may indicate their role in mediating the physiological and pharmacological actions of the EOI. Another way to identify the network of proteins that mediate the actions of the EOI is to exploit methods for identifying interacting proteins. This approach, which is known as proteomics, is well known in the art (see, for example, Blackstock et al. Proteomics: quantitative and physical mapping of cellular proteins. Trends Biotechnol. 17 (3): p. 121-7, 1999; Patton W. F., Proteome analysis II. Protein subcellular redistribution: linking physiology to genomics via the proteome and separation technologies involved. J Chromatogr B Biomed Sci App. 722(1-2):203-23.1999.)

The EOI may exert an effect on the excitability of the target cell. For example, the EOI may block the action of one or more ion channels in the target cell. Methods for modulating the excitability of cells in vitro are well known in the art (Lanigan et al (2001) Biochemistry 40:15528-37; Braun et al (2000) J Physiol. 527:479-92).

The present invention also relates to methods for identifying and/or validating a therapeutic EOI in vivo. Such methods involve the administration of a vector system capable of delivering the test EOI to a subject such that it is delivered to the DRG. The subject is then analysed to see if the EOI has the desired effect in preventing and/or alleviating pain.

Analysis of pain avoidance or relief may be accomplished by a number of known methods. Changes in transmission can be measured by standard electrophysiological techniques in vitro. See for example Hamill et al (1981) Pflugers Arch 391:85-100. Methods for evaluating pain in vivo include the hot plate, tail flick, and formalin tests. See Current Protocols in Neuroscience. Volume 2, Chapter 8.9 Published by John Wiley and Sons.

Preferably the method of the present invention involves an in vitro identification step, followed by an in vivo verification step. Such a method may, for example, involve: (i)

selection of an EOI with therapeutic potential by analysing its effect on an in vitro target cell;

(ii) delivery of the selected EOI to the DRG of a subject; and

(v) analysis of pain in the subject.

This two-step approach facilitated the in vitro screening of a number of candidate drugs, followed by in vivo testing of those which show potential. If the drug then shows promise in the in vivo (for example, animal) screen, then it can go on to clinical trials to assess its usefulness for treating humans and safety.

Pharmaceutical Compositions

The present invention also provides the use of a vector delivery system in the manufacture of a pharmaceutical composition. The pharmaceutical composition may be used to deliver an EOI, such as an NOI, to the DRG of a subject.

The vector delivery system can be a non-viral delivery system or a viral delivery system.

The pharmaceutical composition may be used for treating an individual by gene therapy, wherein the composition comprises or is capable of producing a therapeutically effective amount of a vector system according to the present invention.

The method and pharmaceutical composition of the invention may be used to treat a human or animal subject. Preferably the subject is a mammalian subject. More preferably the subject is a human. Typically, a physician will determine the actual dosage which will be most suitable for an individual subject and it will vary with the age, weight and response of the particular subject.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents that may aid or increase the viral entry into the target site (such as for example a lipid delivery system).

The vector system used in the present invention is preferably administered by direct injection into the subject. In the first preferred embodiment of the invention, where the vector system is administered directly to the DRG of a subject, it is convenient if the system is administered by direct injection to the DRG.

In the second preferred embodiment of the invention (where the vector system is administered to a site which is distant to the DRG and travels to the DRG by retrograde transport) it is convenient if the vector system is injected at the distant site.

The invention will now be further described by way of Examples, which are meant to serve to assist one of ordinary skill in the art in carrying out the invention and are not intended in any way to limit the scope of the invention.

### EXAMPLES

### Example 1

### Expression of Marker Genes in Dissociated and Tissue Explants of Dorsal Root Ganglia

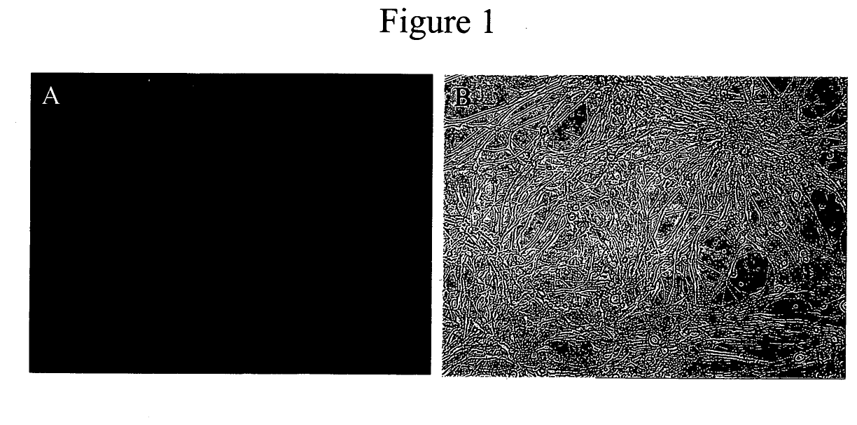

DRGs are isolated from E18 rats according to standard dissection protocols. Cells are dissociated in trypsin for 5 minutes before pelleting down and resuspending in plating media (DMEM+10% FCS, & Gentamycin). Cells are plated out on poly-D-lysine treated glass chamber slides at 1000 cells/mm2 and maintained in a tissue culture incubator at 37 C. for four days. On Day 1 in vitro, cells are transduced with pONY8G,5′ cPPT (a virus capable of expressing green fluorescent protein from a CMV promoter) at an MOI of 10 in neurobasal medium (+B27) for five hours. Polybrene is added to the transduction medium at a concentration of 2 ug/ml−1. After transduction, the media is replaced with fresh neurobasal media and the cells are kept for a further three days. On Day 4 in vitro, transduced cells (GFP positive) are visualized with a microscope equipped with a fluorescent light source and FITC filter set. At least 45% of the cells are transduced and are shown in FIGS. 1a and b.

### Example 2

### Expression of Marker Genes in Dorsal Root Ganglia after Direct Injection of the Virus

Dorsal root ganglia are surgically exposed by dissecting the musculus multifidus and the musculus longissimus lumborum and by removing the processus accessorius and parts of the processus transversus. An EIAV vector coding for the reporter gene β-gal is injected directly in the DRG. Subjects will receive 1 μl of the viral solution per ganglion. All injections are done by using a stereotaxic frame and a Hamilton syringe with 34-gauge needle. The solution is slowly infused at the speed of approximately 0.1 μl/min. To determine the transduction efficiency of EIAV vector for sensory neurons, histology and immunohistology using β-gal antibodies (Affiniti) is performed at different time points.

### Example 3

### Expression of Marker Genes in Dorsal Root Ganglia after Peripheral Administration of the Virus

The procedure of the application of the virus on the skin surface has been described previously (Wilson et al., 1999 Proc Natl Acad Sci USA, 96(6):3211-3216.). Briefly, the hair is removed from the dorsal of the hindfoot surface. The skin is scarified using medium-coarse sandpaper. Ten microliters of the viral solution is applied to each foot. The side of a pipettor tip is used to spread the virus. Alternatively, or additionally, the virus can be injected into the footpad. The virus is retrogradely transported to the DRG and can be detected using β-gal antibodies (5′-3′).

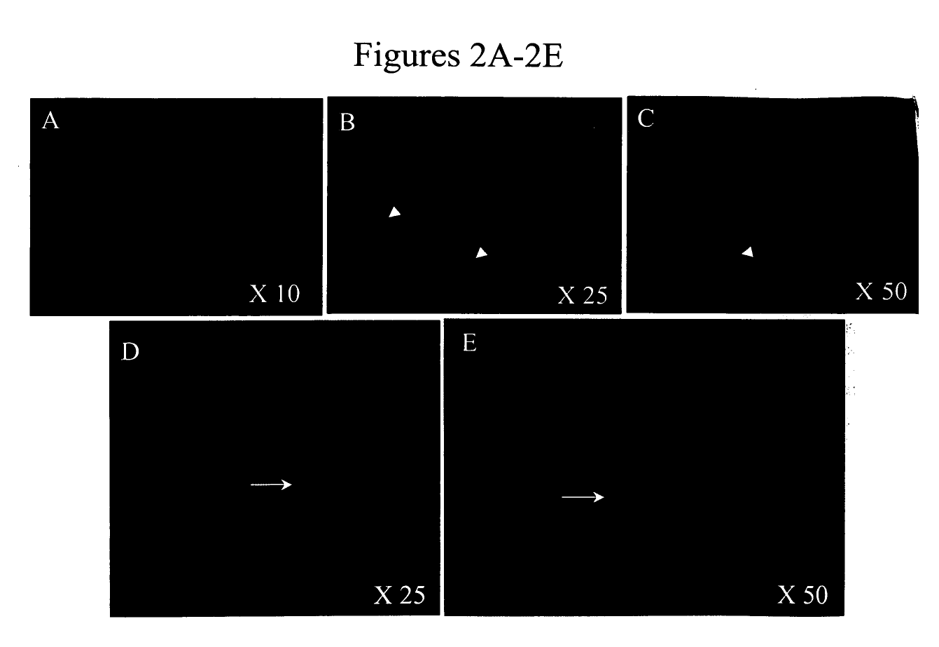

pONY8Z vectors were injected into the footpads of 8 rats and analysed 4 weeks post-transduction (rabies-G 6×10⁸ TU/ml (20 μl), n=4; VSV-G 6×10⁸ TU/ml (20 μl), n =4). Rabies-G pseudotyping confers retrograde transport of the viral vector (see Mazarakis et al., 2001. Human Molecular Genetics, 10:2109). Histological sections from the DRG at ×40 magnification were examined. All animals displayed retrogradely transduced DRG neurons (FIG. 2A-E). However, in contrast to pONY8Z rabies-G injected rats, no β-gal reactivity was detectable in DRG sections from rats injected with pONY8Z VSV-G.

### Example 4

### Expression of Marker Genes in Dorsal Root Ganglia after Direct Injection Into the Spinal Cord

Group of rats are injected with pONY8Z (rabies-G or VSV-G) or equivalent amount of PBS, via a posterior laminectomy within the dorsal horn of the spinal cord. Three injection sites at the lumbar level, separated by 2 mm, are performed. Each rat received 1 μl per site of the viral solution at dorso-ventral coordinate of 0.5 mm.

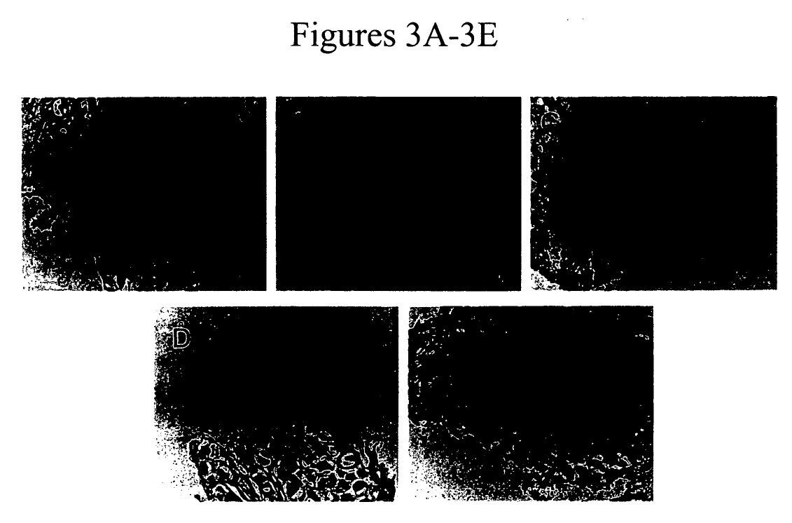

pONY8Z vectors were injected into the dorsal horn in four rats and analysed 5 weeks post-transduction (rabies-G 3.8×10⁸ TU/ml, n=2; VSV-G 1.2×10⁹ TU/ml, n=3). Rabies-G pseudotyping confers retrograde transport of the viral vector (see Mazarakis et al., 2001. Human Molecular Genetics, 10:2109). Histological sections from the spinal cord, the dorsal root and DRG were examined at various magnifications. All animals showed expression of the marker gene to the immediate neighbourhood of the site of injection into the spinal cord. Of 3 rats injected into the spinal cord with pONY8Z rabies-G, 2 showed expression of β-gal in Schwann cells. Axonal expression also was seen (FIG. 3A-C). The two rats displayed retrogradely transduced DRG neurons (FIG. 3D-E). However, in contrast to pONY8Z rabies-G injected rats, no β-gal reactivity was detectable in dorsal root and DRG sections from rats injected with pONY8Z VSV-G.

### Example 5

### Expression of Potassium Channels in Dissociated Dorsal Root Ganglion Cells

Dissociated DRG cells are prepared as described in example 1. On Day 1 in vitro, cells are transduced with (Smart2Kir_IRES_GFP), av virus encoding the inward rectifier potassium channel (KIR6.2) and GFP. On DIV4 cells are visualized under fluorescent light and are used for patch clamp electrophysiology according to standard patch clamp electrophysiology procedures. Results show that cells transduced with virus have lower resting membrane potentials (hyperpolarisation).

### Example 6

### Expression of Potassium Channels in Dorsal Root Ganglia after Direct Injection

(Smart2Kir_IRES_GFP) is directly injected into rat DRG following the procedure of Example 2. Transduction is assessed using either by GFP fluorescence or antibody staining.

### Example 7

### Expression of Potassium Channels in Dorsal Root Ganglia after Peripheral Delivery

(Smart2Kir_IRES_GFP) is injected into rats by subcutaneous injection in the hindfoot as described in example 3. Transduction is assessed using either GFP fluorescence or antibody staining.

### Example 8

### Expression of Antisense Message Specific for Sodium Channels in DRG Neurons after Injection Into the Footpad

Twenty microliters of the viral vectors (Smart2antisense-flag) is applied to each footpad. The virus is retrogradely transported to the DRG and can be detected using antibodies against Flag (Sigma).

### Example 9

### Expression of Reporter Gene in DRG Neurons after Intrasciatic Injection

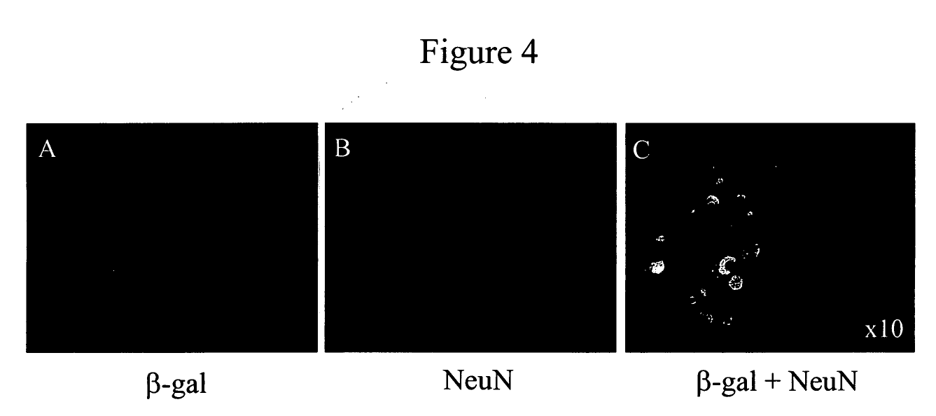

Adult rats (n=3) were anaesthetized and injected with an pONY8Z Rabies-G pseudotyped viral vector (2 μl) into the sciatic nerve. Five weeks following sciatic injection of the viral vector, animals were sacrificed and dorsal root ganglia removed. Tissue was sectioned and immunolabelled for NeuN and for β-galactosidase. In order to visualize staining, Cy3 anti-mouse and Alexa-488 anti-rabbit secondary antibodies against the NeuN and β-galactosidase primary antibodies were used (FIGS. 4A and B). Double labelling of neuronal bodies in the dorsal root ganglia is demonstrated by colocalization of the two markers in yellow (FIG. 4C).

### Example 10

### Expression of Antisense Message Specific for Sodium Channels in DRG Neurons after Intrasciatic Injection

For intranerval injection, the right sciatic nerve of anaesthetized rat is surgically exposed. The nerve was gently placed onto a metal plate and Smart2Z or Smart2antisensesodimchannel-flag pseudotyped with rabies-G envelope are injected with a 33-gauge Hamilton syringe over 3 min. The volume injected per rat is 2-3 μl. The sciatic nerve is anatomically repositioned, and the skin was closed with vicryl 5/0 sutures.

### Example 11

### Expression of Neurotrophic Factor in the DRG Neurons after Intrasciatic Injection

Intranerval injection of vectors expressing neurotrophic factors is performed as described in example 10.

### Example 12

### Screening of a Plurality of Test Compounds In Vitro and In Vivo

The gene encoding the calcium sensing protein DREAM is expressed in an EIAV viral vector in cultured dissociated dorsal root ganglia. Cells are dissected, cultured and transduced as described in previous examples. After transduction, RNA is isolated and compared with cells transduced with an empty viral vector. RNA are analysed using the Affymetrix chip system. Similarly, cells are transduced with a viral vector expressing a constitutively active version of the immediate upstream activator of both ERK1 and ERK2, mitogen-activated/extracellular signal-regulated kinase 1 (MEK1), to activate ERK signalling. Again by comparison of RNA using the Affymetrix system, changes in gene expression as a result of physiological processes that are associated with modulation of pain are analysed.

The invention is further described by the following numbered paragraphs.

1. A method for treating and/or preventing pain in a subject, which comprises a step of administration of a lentiviral vector system such that it delivers an EOI to a DRG of the subject.

2. A method according to paragraph 1, in which the vector system is administered by injection into a DRG of the subject.

3. A method according to paragraph 1, wherein

(i) the vector system is administered to the subject at a site which is distant to the DRG

(ii) the vector system or part thereof travels to the DRG by retrograde transport.

4. A method according to paragraph 3, wherein vector system is or comprises at least a part of a rabies G protein or a mutant, variant, homologue or fragment thereof.

5. A method according to paragraph 3 or 4, wherein the administration site is a peripheral site.

6. A method according to paragraph 3, 4 or 5, wherein the vector system is administered to the subject by injection into the area of pain.

7. A method according to any preceding paragraph, wherein the EOI is capable of modulating the cellular excitability of a target cell.

8. A method according to paragraph 7, wherein the EOI is capable of causing hyperpolarisation of the target cell.

9. A method according to any preceding paragraph, wherein EOI is capable of modulating the expression and/or activity of an ion channel.

10. A method according to paragraph 9, wherein EOI is capable of causing the expression of an ion channel or part thereof.

11. A method according to paragraph 10, wherein the ion channel is constitutively active.

12. A method according to any preceding paragraph, in which vector system

(i) the EOI is an NOI;

(ii) expression of the NOI is under the control of a targeted promoter; and

(iii) the targeted promoter restricts the expression of the NOI to C fibers and/or A* fibres.

13. A method according to any of paragraphs 1 to 11, in which vector system

(i) the EOI is an NOI; and

(ii) expression of the NOI is inducible.

14. A method according to any preceding paragraph, wherein the EOI is delivered to the cell body of a sensory neuron within the DRG.

15. The use of a vector system as defined in any preceding paragraph in the manufacture of a pharmaceutical composition to treat and/or prevent pain in a subject, wherein, in use, the pharmaceutical composition is administered such that the EOI is delivered to a DRG of the subject.

16. A method for delivering an EOI to the spinal cord, which comprises the following steps:

(i) delivery of an EOI to the cell body of a sensory neuron using a method according to paragraph 14;

(ii) optional modification of the EOI; and

(iii) delivery of the optionally modified EOI from the cell body of the sensory neuron to the spinal cord via the central branch of the sensory neuron.

17. A method for identification and/or validation of an EOI useful in the prevention and/or treatment of pain which comprises the step of

(i) delivery of a test EOI to target cell;

(ii) analysis of the effect of the EOI on the target cell; and

(iii) selection of an EOI with therapeutic potential.

18. A method according to paragraph 17, wherein step (ii) comprises monitoring EOI-induced modulation of the transcriptome and/or proteosome of the target cell.

19. A method according to paragraph 17 or 18, wherein the target cell is derivable from a DRG.

20. A method according to any of paragraphs 17 to 19, wherein the target cell is in vitro.

21. A method according to paragraph 19, wherein the target cell is in situ within the DRG.

22. A method according to paragraph 21, which comprises the following steps:

(i) administration of a vector system such that it delivers an EOI to a DRG of a subject by the method as described in any of paragraphs 1 to 14; and

(ii) analysis of pain in the subject.

23. A method according to paragraph 22, wherein the perception and/or transmission of pain in the subject is analysed.

24. A method according to paragraph 22 or 23, which comprises the following steps:

(i) in vitro selection of an EOI with therapeutic potential by a method according to paragraph 20;

(ii) delivery of the selected EOI to the DRG of the subject; and

(v) analysis of pain in the subject.

25. An EOI useful in the prevention and/or treatment of pain identified or validated by a method according to any of paragraphs 17 to 24.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biology or related fields are intended to be within the scope of the following claims.

We claim:

1. A method for treating or preventing pain in a subject, comprising administering a lentiviral vector system comprising an entity of interest (EOI) to a dorsal root ganglion cell (DRG) in the subject.

2. The method according to claim 1, wherein the vector system is administered by injection into a DRG of the subject.

3. The method according to claim 1, wherein the vector system is administered to the subject at a site which is distant to the DRG and the vector system or a part thereof travels to the DRG by retrograde transport.

4. The method according to claim 3, wherein the vector system is or comprises at least a part of a rabies G protein or a mutant, variant, homologue or fragment thereof.

5. The method according to claim 3, wherein the site is a peripheral site.

6. The method according to claim 3, wherein the vector system is administered to the subject by injection into an area of pain.

7. The method according to claim 1, wherein the EOI modulates cellular excitability of a target cell.

8. The method according to claim 7, wherein the EOI causes hyperpolarisation of the target cell.

9. The method according to claim 1, wherein the EOI modulates expression or activity of an ion channel.

10. The method according to claim 9, wherein the EOI causes expression of an ion channel or part thereof.

11. The method according to claim 10, wherein the ion channel is constitutively active.

12. The method according to claim 1, wherein:
(i) the EOI is an NOI;
(ii) expression of the NOI is under the control of a targeted promoter; and
(iii) the targeted promoter restricts the expression of the NOI to C fibers and/or A* fibres.

13. The method according to claim 1, wherein:
(i) the EOI is an NOI; and
(ii) expression of the NOI is inducible.

14. The method according to claim 1, wherein the EOI is delivered to a sensory neuron cell body within the DRG.

15. The method according to claim 14, wherein the EOI is optionally modified in the sensory neuron cell body and is delivered from the sensory neuron cell body to the spinal cord via the central branch of the sensory neuron.

16. A method for identification or validation of an EOI useful in the prevention or treatment of pain comprising
(i) delivering a test EOI to target cell;
(ii) analyzing the effect of the EOI on the target cell; and
(iii) selecting an EOI with therapeutic potential.

17. The method according to claim 16, wherein step (ii) comprises monitoring EOI-induced modulation of a transcriptome and/or proteosome of the target cell.

18. The method according to claim 16, wherein the target cell is derived from a DRG.

19. The method according to claim 16, wherein the target cell is in vitro.

20. The method according to claim 18, wherein the target cell is in situ within the DRG of a subject.

21. The method according to claim 20, wherein step (ii) comprises analysis of pain in the subject.

22. The method according to claim 21, wherein perception and/or transmission of pain in the subject is analysed.

23. The method according to claim 20, further comprising:
(iv) delivering the EOI to a DRG of the subject; and
(v) analyzing pain in the subject.

24. An EOI useful in the prevention or treatment of pain, wherein the EOI is identified or validated by the method according to claim 16.
